(19) United States
(12) Patent Application Publication
(10) Pub. No.: US 2005/0266076 A1
(43) Pub. Date: Dec. 1, 2005

(54) Resorbable delivery systems for the treatment of cancer

(75) Inventor: Garfield P. Royer, Frederick, MD (US)

Correspondence Address: NIXON & VANDERHYE, PC, 901 NORTH GLEBE ROAD, 11TH FLOOR, ARLINGTON, VA, 22203, US

(73) Assignee: Royer Biomedical, Inc., Frederick, MD (US)

(21) Appl. No.: 10/518,034

(22) PCT Filed: Jun. 17, 2003
(86) PCT No.: PCT/US03/19007

§ 371 Date: Dec. 14, 2004

Related U.S. Application Data
(60) Provisional application No. 60/389,934, filed on Jun. 20, 2002.

(51) Int. Cl. A61K 33/24; A61K 9/22

(52) U.S. Cl. 424/468; 424/649; 514/49

(57) ABSTRACT

This invention relates generally to the production and use of inorganic-polymer complexes for the controlled release of anti-cancer agents. Advantageously, the inorganic used is calcium sulfate. The invention also includes novel methods of treating cancer.

### FIELD OF THE INVENTION

This invention relates to the general area of resorbable drug-delivery systems and their use in treating cancer. Sustained, localized release of agents useful in treating cancer, such as an antineoplastic agent and an immunostimulant, regionally or directly within the tumor improves outcomes and reduces toxicity of the agents.

### BACKGROUND OF THE INVENTION

Anti-tumor agents are typically given systemically by intravenous delivery. The toxicity, which can be severe, involves myelosuppression, nausea, rashes, diarrhea, and hair loss. Antineoplastic agents typically lack specificity. Although these chemical substances will preferentially kill rapidly dividing cells, they do not distinguish between normal cells and cancerous cells. Hence, cells of hair follicles are killed which results in alopecia; destruction of bone marrow cells results in degradation of immune capability and anemia; cells of the epithelial lining are also destroyed. Hepatic, neurologic, renal, and cardiac toxicities are of concern with many antineoplastic agents.

Polymer matrices designed for controlled release of bioactive compounds can be non-resorbable or resorbable. In general, resorbable means degradable in the body by erosion from the surface or breakdown from within. The mechanism can involve either a chemical reaction, such as hydrolysis, or dissolution.

Resorbable polymer matrices for controlled release are usually based on an oxygenated monomer, which is condensed in organic solvent to yield the water-insoluble polymeric product. The bioactive agent and the polymer are then combined in such a way as to give a timed-release formulation. The combination of active ingredient and polymer often involves organic solvents as well. The use of organic solvents is a decided disadvantage, especially when large-scale production is required. Toxic residues of organic solvents are a concern. Proteins (such as cytokines) and many polypeptides are incompatible with organic solvents. The types of polymers in this category include:
polyesters
polyanhydrides
polyketals
poly(orthoesters)
polyurethanes (Burkersroda, F V and Goepferich, A M in Biomedical Materials, T Neenan, M Marcolongo and R F Valentini, eds. (1999), page 23, Materials Research Society, Warrendale Pa.) Naturally occurring proteins may be used as structural components in drug-delivery matrices (Royer, G P U.S. Pat. No. 4,349,530; Royer, G P U.S. Pat. No. 5,783,214; Lee, T K, et al., Science (1981) 233-235). One deficiency of proteinaceous delivery matrices is that they can exhibit instability especially in environments such as a necrotic tumor or where an inflammatory reaction is present.

Commonly owned U.S. Pat. No. 6,391,336 and WO 99/15150 disclose stable, yet practical compositions for use in inflamed sites comprising an inorganic compound, a matrix polymer and/or a complexing agent. These compositions have the advantage of being biocompatible but, unlike synthetic organic polymers, no non-aqueous solvents are required in the preparation. The drug is incorporated as a solid or as part of the matrix polymer solution. The material can also be used as a slurry, that is, it can be injected directly into a lesion and allowed to solidify in situ.

Commonly owned U.S. Ser. No. 09/703,710 discloses an inorganic delivery system with a conditioning agent.

### OBJECT OF THE INVENTION

It is an object of this invention to provide a safe, resorbable delivery system that can be designed and fashioned to provide controlled (sustained), localized release of antineoplastic agents, radiation potentiators, apoptosis inducers, immunostimulants, radioisotopes and other active anti-tumor compounds/entities over a predetermined time-course.

It is a further object of this invention to provide new methods of treating cancer using sustained release formulations.

### SUMMARY OF THE INVENTION

The subject invention relates to a method of treating a solid tumor in a mammal comprising: administering to said mammal a resorbable delivery system for sustained release of i) an antineoplastic agent and ii) an immunostimulant.

The subject invention includes a composition for the controlled release of an antineoplastic agent and an immunostimulant comprising an antineoplastic agent and an immunostimulant dispersed throughout a matrix wherein said composition is the hydration reaction product of an aqueous mixture comprised of:
an inorganic compound capable of undergoing hydration and/or crystallization,
an antineoplastic agent,
an immunostimulant, and
at least one of: a matrix polymer, a complexing agent, and a conditioning agent.

The subject invention also relates to a method of treating a solid tumor in a mammal comprising:
a) administering to said mammal a resorbable delivery system for sustained release of a radiation potentiator, and
b) irradiating said tumor.

The subject invention includes a composition for the controlled release of a radiation potentiator comprising a radiation potentiator dispersed throughout a matrix wherein said composition is the hydration reaction product of an aqueous mixture comprised of:
an inorganic compound capable of undergoing hydration and/or crystallization, a radiation potentiator, and
at least one of: a matrix polymer, a complexing agent, and a conditioning agent.

Additionally, the invention includes a method of treating a tumor in a mammal comprising:
a) treating ex vivo tumor cells from said mammal with an antineoplastic agent, and
b) administering to said mammal said tumor cells treated in step a) and
c) administering to said mammal a resorbable delivery system for sustained release of an immunostimulant.

### DETAILED DESCRIPTION OF THE INVENTION

The subject invention relates to drug-delivery systems that are based on resorbable matrices and include agents for treating cancer. The systems are safe, resorbable and can be designed and fashioned to provide controlled, localized release of antineoplastic agents, radiation potentiators, apoptosis inducers, immunostimulants, and other active anti-tumor compounds/entities such as genetic constructs that encode immunostimulants or anti-tumor proteins, over a pre-determined time-course. Sustained, localized release of antineoplastic agents regionally or directly within the tumor improves the outcome of the cancer treatment and reduces toxicity of the antineoplastic agent. The matrix is capable of providing sustained release over a pre-determined time period.

The invention also provides new methods of treating cancer using sustained release formulations. Antineoplastic agents such as cisplatin, paclitaxel, and doxorubicin can be delivered directly to a cancerous lesion or to the tumor vasculature. Combination with an immunostimulant is useful in treatment of local and remote tumors. Localized delivery of a radiation potentiator can also be done. The reduction of systemic toxicity is a decided benefit with these therapeutic modalities. Localized delivery of paclitaxel is advantageous in that toxicity is reduced and paclitaxel can function as a radiation potentiator as well as an anti-cancer agent. Immunostimulants such as GM-CSF are effective when delivery locally in tandem with antineoplastic agents. Co-administration of immunostimulants is an advantageous embodiment. Use of localized delivery of paclitaxel is beneficially combined with radiation therapy.

Localized delivery of TNF-alpha, IL-2 or an anti-angioplastic agent such as endostatin or angiostatin, alone or in combination with an antineoplastic agent is also effective.

### A. INTRODUCTION

Entrapment of bioactive substances within the resorbable biocompatible matrix described herein yields a delivery system, which permits controlled and localized release of anticancer agents. Specifically, antineoplastic agents, immunostimulants, and radiation potentiators can be delivered locally which enhances efficacy and reduces toxicity. Matrix beads or granules containing these active ingredients can be mixed in varying proportions prior to installation in a tumor. Inorganic compounds such as CaSO₄-½ H₂O (calcium sulfate hemihydrate) can be combined with a polymer in the presence of a bioactive agent to produce a solid which constitutes a biocompatible and resorbable delivery matrix (See WO 99/15150 and U.S. Pat. No. 6,391,336—hereby incorporated by reference in their entirety). The matrix polymer increases the internal viscosity of the device, which slows the efflux of the bioactive agent.

Microgranules (45-150 microns), 3 mm spherical beads, and other molded structures can be produced. The production of the delivery system can be illustrated as follows:

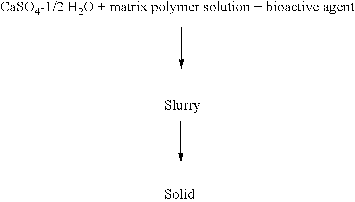

When contacted with water calcium sulfate hemihydrate is converted to the dihydrate, CaSO₄-2 H₂O, which crystallizes. The mass of needle-like crystals produces a porous matrix with high compressive strength, as much as 2000 psi or more. A conditioning agent such as calcium stearate can be pre-mixed with the calcium sulfate hemihydrate. The slurry can be injected into the desired location e.g. directly into a lesion, with solidification in situ. This form is termed matrix slurry. The fact that the slurry can set-up in the presence of moisture is advantageous. The matrix slurry containing anti-tumor agents can be installed in cavities left by surgery to prevent recurrence.

The delivery matrix includes:
a. an inorganic compound capable of undergoing hydration and/or crystallization, plus
b. a matrix polymer, and/or
c. a complexing agent, and/or
d. a conditioning agent, which improves stability, extends the residence time, and provides for control of the release profile.

The nature and amount of matrix polymer, the relative proportions of calcium sulfate hemihydrate and liquid, the complexing agent, and the nature and amount of the conditioning agent permit the adjustment of the release profile and residence time.

The use of a conditioning agent provides added control of the release profile and residence time. In addition, it imparts the desirable feature of moisture resistance, which preserves the shape of the mass while setting. When matrix slurry containing calcium stearate is submerged after thorough blending, the mass remains intact and setting occurs. This attribute is very important as it allows the installation of matrix slurry into moist areas of the body. Water repulsion can also stabilize the solid dosage form with extension of residence time. Calcium stearate is included at a rate of 2.5-30%, advantageously 5-25% w/w based on the amount of calcium sulfate hemihydrate. Even higher levels of calcium stearate are obtainable depending on the nature and amounts of other components.

The material of the invention has the advantage of being biocompatible but, unlike synthetic organic polymers, non-aqueous solvents are not required in the preparation. The drug is incorporated as a solid or as part of the matrix polymer solution. The preparation entails
a. mixing of the inorganic powder and the matrix polymer solution,
b. solidification, and
c. molding or milling.

For use in regional delivery, microgranules (45-150 microns) can be introduced into the arterial blood supply of the tumor. Agents useful in treating cancer are then delivered. Microgranules can be used postoperatively to prevent growth of remnant malignant cells/tissues missed during surgical resection. Beads (1-3 mm) containing agents useful in treating cancer can be surgically placed directly into the tumor. The dimensional and chemical stability of the matrix are advantages compared to proteinaceous gels for example. These gels are mobile and susceptible to enzymatic attach; the matrix of the invention is less mobile and not susceptible to proteolysis.

### B. PRODUCTION OF DOSAGE FORMS

The following process achieves the production of the delivery matrix:
a. blending of calcium sulfate hemihydrate and conditioning agent such as calcium stearate, both in powder form;
b. mixing with polymer solution. Drug can be dissolved or suspended in the matrix polymer solution;
c. solidification in a mold or in bulk; and
d. unmolding or preparing of microgranules by milling and sizing.

The molds made of stainless steel or Teflon can be used to prepare cylinders or spheres, both 3 mm in diameter. The preparation of wafers is also possible. Microgranules can in turn be compressed into tablets with various binding agents to yield another dosage form.

A representative formulation follows:

| Ingredient | Amount | |
|---|---|---|
| Calcium sulfate hemihydrate | 0.9 | g |
| Calcium stearate | .1 | g |
| PEG (10% w/v) | 0.6 | ml |
| Paclitaxel | .05 | g |

When the amount of calcium sulfate hemihydrate is set at about 1 g the amount of bioactive substance is typically set in the range of 1-300 mg. The concentration of polymer can be as high as 50% (w/v). The conditioning agent is present in the range of 2.5-30% (w/w) based on calcium sulfate. The ratio of liquid/solid is advantageously 0.6.

The calcium sulfate hemihydrate can be sterilized by dry heat (140° for 4 hr); the polymer solution is sterilizable by filtration (0.2-micron filter). Terminal sterilization by gamma irradiation at 15-18 kGy is also effective.

The components of the compositions of the invention are described below.

1. Inorganic Compounds

Calcium sulfate hemihydrate is an advantageous inorganic component. The hemihydrate takes up water and crystallizes as the higher hydrate. Unadulterated calcium sulfate matrix exhibits poor drug release profiles. With conditioning agents, and optionally matrix polymers and complexing agent-active agent complexes the release profiles are improved. Other inorganics can be employed such as calcium silicates, aluminates, hydroxides and/or phosphates (see pages 72, 95, 327 in Reference Book of Inorganic Chemistry (1951) Latimer, W. H., and Hildebrand, J. M., Macmillan, New York, hereby incorporated by reference in its entirety).

2. Matrix Polymers

The preferred matrix polymers for medical use are
biocompatible (non-toxic, non-allergenic, non-immunogenic)
water soluble
compatible with other components in the formulation

Examples of matrix polymers include chondroitin sulfate, hyaluronic acid, dextran sulfate, pentosan polysulfate, polyethylene glycol (PEG), polyvinylpyrrolidone (PVP), proteins such as gelatin and fibrinogen. Counterions, are advantageously sodium or calcium. Chitosan as well as cationic polypeptides, polylysine, and polyarginine are examples of useful matrix polymers that are positively charged at neutral pH. See commonly owned WO 99/15150, U.S. Pat. No. 6,391,336 and U.S. Ser. No. 09/703,710 hereby incorporated by reference in their entirety.

The function of the matrix polymer is to control the viscosity, which is dependent on the nature, molecular weight and concentration of the polymer. The rationale for using polymers and polymeric complexing agents is based on Stokes law:
D ∝ 1/Mv
D=the diffusion coefficient
M=the molecular weight of the medicinal
v=the viscosity of the medium

3. Complexing Agents

To the extent that polymeric complexing agents increase the effective molecular weight of the active ingredient, the rate of efflux would be slowed according to D ∝ 1/Mv. Complexing agents can be polymers or small molecules. The agents can form ionic bridges or hydrophobic bonds with the molecule to be delivered. The complexes involving the bioactive agents can range from sparingly soluble to soluble. Disodium pamoate is a good example of a complexing agent that forms sparingly soluble adducts with cationic bioactive ingredients. Disodium methylene disalicylate is a similar molecule that performs the same function. Procaine and benzathin can be used to reduce the solubility and rate of efflux of anionic bioactive agents. See commonly owned WO 99/15150, U.S. Pat. No. 6,391,336 and U.S. Ser. No. 09/703,710.

4. Conditioning Agents

Conditioning agents are used to slow the erosion rate and permit solidification in the presence of moisture. Calcium stearate is a practical choice that meets the criteria of safety and efficacy. Other calcium salts are useful in this regard. Examples include saturated and unsaturated carboxylic acids, aromatic carboxylic acids, corresponding phosphates, phosphonates, sulfates, sulfonates, and other compounds containing a hydrophobic moiety with a negatively charged anion. Salts of undecylenic acid are useful.

The use of calcium as the cation is preferred but other cations will suffice; the group includes, but is not limited to, zinc, magnesium, aluminum and manganese.

See commonly owned U.S. Ser. No. 09/703,710.

5. Coatings

The compositions of the subject invention can be coated as disclosed in U.S. Provisional Application Ser. No. 60/389,933 filed Jun. 20, 2002 and entitled “RESORBABLE MATRICES WITH COATINGS FOR DELIVERY OF BIOACTIVE COMPOUNDS” (Attorney Dkt. No. 1729-25), the entire content of which is expressly incorporated hereinto by reference.

6. Agents Useful in the Treatment of Cancer

a. Antineoplastic Agents

Localized delivery of the anti-tumor agent results in reduction or elimination of the systemic toxicity.

Antineoplastic agents useful in the compositions of the subject invention include carmustin, paclitaxel, doxorubicin, cisplatin, ifosfamide, cytoxan, carboplatin, methotrexate, leuprolide, bleomycin, and fluorouracil (e.g. 5-FU). As used herein, the term antineoplastic agent includes apoptosis inducers. Apoptosis inducers are also useful in the subject invention. See Clinical Oncology (2001) Philip Rubin, Ed. pages 41 and 86, W B Saunders, Philadelphia.

Localized delivery of antineoplastic agents can be combined with systemic administration of the same or different antineoplastic agent. In one embodiment, the systemic administration is with a less toxic antineoplastic agent such as anti-metabolites (5FU, methotrexate) or anti-angioplastic agents such as endostatin or angiostatin. Synergistic drug action plus reduction in the probability of tumor cells becoming tolerant are advantages to this approach.

b. Immunostimulants

Immunostimulants, compounds that stimulate an immune response against tumor cells, useful in the subject invention include the following: lipopolysaccharide (LPS), Bacille Calmette-Guerin (BCG), keyhole limpet hemocyanin (KLH), interferons such as alpha or beta interferon, cytokines such as interleukins e.g.s IL-1, IL-2, IL-6, colony stimulating factors e.g.s G-CSF and GM-CSF, and TNF-alpha.

In another embodiment, a genetic construct encoding an immunostimulant is substituted for the immunostimulant.

c. Radiation Potentiators

There is similar justification for localized administration of radiation potentiators, i.e. compounds that enhance the anti-tumor effects of radiation. The focus of radiation to a specific area where the radiation potentiator is present in high concentration improves efficacy and reduces side effects.

Radiation potentiators useful in the subject invention include compounds such as paclitaxel, nimorazole, metronidazole, and 5,6-dimethylxanthenone-4-acetic acid and related compounds that have similar activity.

d. Radioisotopes

Radioisotopes useful in the subject invention include Pd-103, I-125, and Ir-192 or other isotopes currently used in implants for treatment of cancer.

C. USES OF AND ADMINISTRATION OF THE COMPOSITIONS OF THE INVENTION

Treatment Modality

Each of the treatment modalities discussed below utilizes a resorbable matrix for sustained release of an agent. Although the inorganic based matrix delivery system discussed above is advantageous, the treatment modalities are not limited to the use of such matrices. As used herein the term “therapeutically effective amount” is the amount of agent sufficient to maintain or reduce the size of the tumor. As used herein, “mammal” includes humans, horses, dogs, cats and other mammalian pet animals. As used herein “sustained release” means a few days e.g. 2, 3 or 4, or weeks e.g. 1-3 weeks, up to 8 weeks.

1. Antineoplastic Agent Plus Immunostimulant

The simultaneous localized delivery of an antineoplastic agent and an immunostimulant produces a response because of the activation of antigen presenting cells such as dendritic cells. Tissue destruction coupled with sustained levels of immunostimulants (below) allows the body to mount an immune response against the tumor. Also, localized delivery of antineoplastic agent in combination with an immunostimulant strengthens rather than degrades the immune system and improves the chance that the patient might mount an immune response against the tumor. Thus, the patient is better able to marshal natural defenses against the malignancy.

In one embodiment of the invention, a resorbable delivery system for sustained release of an antineoplastic agent and a resorbable delivery system for sustained release of an immunostimulant are co-administered to a mammal e.g. via intratumoral implantation.

In a further embodiment, a delivery system for sustained release including an apoptosis inducer is installed with a delivery system for sustained release including an immunostimulant. Alternatively, in each of the above embodiments, the active agents can be included in the same resobable sustained delivery system.

2. Radiation Potentiator Plus External Radiation

In another embodiment, a resorbable delivery system for sustained release of a radiation potentiator (such as paclitaxel) is administered to a mammal e.g. intra-tumorally. After installation of the delivery system containing the radiation potentiator, the tumor is irradiated. In the case of a delivery system containing paclitaxel, there is benefit with regard to chemical toxicity and radiation toxicity to the tumor with minimal systemic toxicity.

3. Radiation Potentiator Plus Radioisotope (Internal Radiation)

Another embodiment comprises administering (intra-tumoral/peri-tumorally) to a mammal a resorbable delivery system for sustained release of a radiation potentiator in conjunction with a radioisotopic implant, e.g. a resorbable delivery system for sustained release of a radioisotope (such as Pd-103, I-125, Ir-192 or other isotopes currently used in implants for treatment of cancer).

### D. SITE OF DELIVERY AND ADMINISTRATION

The development of improved sonographic guidance systems enable the use the treatment modalities described above with minimal trauma and cost. Installation can be achieved by direct injection when the tumor is accessible or by cannula or endoscope using ultrasonic guidance.

In an important embodiment, a delivery system for sustained release including the agent useful in treating cancer (eg antineoplastic agent) is installed at a site discussed below, e.g. intra-tumorally/peri-tumorally, along with the same or different agent useful in treating cancer given systemically with or without a sustained release delivery system.

1. Intra-Tumorally/Peri-Tumorally

In an advantageous embodiment of the invention, a delivery system including an agent useful in treating cancer (antineoplastic agent, immunostimulant, radiation potentiator, radioisotope, or apoptosis inducer) is installed intra-tumorally/peri-tumorally only.

The compositions of the subject invention are particularly well suited for intra-tumoral/peri-tumoral delivery in treating specific types of solid tumors including: squamous cell carcinomas, melanomas, lymphomas, sarcomas, sarcoids, osteosarcomas, tumors associated with skin cancer, breast cancer, head and neck cancer, gynecological cancer, urological and male genital cancer, bladder cancer, prostate cancer, bone cancer, cancers of the endocrine glands, cancers of the alimentary canal (e.g. colon cancer), cancers of the major digestive glands/organs (e.g. stomach, liver, pancreas), CNS cancer (including brain cancers such as a gliomas), and lung cancer.

The form of the delivery system used for intra-tumoral/peri-tumoral delivery is typically matrix beads (1-3 mm which can be administered by arthroscopic cannula) or microgranules (advantageously 45-150 microns which can be administered by using e.g. a 3% hyaluronic acid solution for suspending the microgranules).

2. Tumor Vasculature

In another embodiment, the delivery system for sustained release including the agent useful in treating cancer (as described above for intra-tumorally/peri-tumorally) is injected into the tumor vasculature.

The form of the delivery system used for tumor vasculature delivery is typically matrix beads (1-3 mm which can be administered by arthroscopic cannula) or microgranules (advantageously 45-150 microns which can be administered by using e.g. a 3% hyaluronic acid solution for suspending the microgranules).

3. Cavities Left by Tumor Resection

A delivery system for sustained release including an agent useful in the treatment of cancer can be installed in cavities left by tumor resection (in the form of e.g., a suspension of microgranules using e.g. a 3% hyaluronic acid solution). To prevent recurrence of breast cancer following breast-conserving lumpectomy, patients are often subjected to multiple radiation treatments. This demanding treatment can be replaced by a treatment using a delivery system for sustained release including an agent useful in the treatment of cancer such as paclitaxel. The filling of such cavities following lumpectomy in breast cancer treatment is advantageous. As paclitaxel is also a radiation potentiator, the procedure can be combined with local radiation for added protection and minimal toxicity. A less toxic anti-metabolite, such as 5-FU can be given systemically for protection against distant metastasis.

The form of the delivery system used for cavities left by tumor resection delivery is typically matrix beads (1-3 mm which can be administered by cannula) or microgranules (advantageously 45-150 microns).

4. Lung Cancer

In another embodiment involving lung cancer, a nebulizer is used to administer the sustained delivery system e.g. microgranules including the agent useful in treating cancer such as paclitaxel. The tumor is optionally subsequently irradiated.

Ex vivo Treatment of Tumor Cells

In a still further embodiment, there is ex vivo treatment of tumor cells with an antineoplastic (e.g. cancer cells are treated with cisplatin in vitro). The suspension of treated cells is then reintroduced plus sustained release delivery system including an immunostimulant (e.g. GM-CSF in the form of microgranules). The suspension and the sustained release delivery system including the immunostimulant are then administered (e.g.s injected subcutaneously or introduced into the cavity left by surgery e.g. using 3% hyaluronic acid solution for suspending the microgranules). Use of an immunostimulant of microbiological origin (such as LPS) in addition to, or as an alternative to GM-CSF is also an option. Other cytokines (interleukins, colony stimulating factors) can be used alone or in combination with GM-CSF. Alternatively, the treated cells can be mixed with an immunostimulant and formulated as a delivery system for sustained release.

The form of delivery system used for delivery of the above described is typically matrix beads (1-3 mm which can be administered by arthroscopic cannula) or microgranules (advantageously 45-150 microns).

The following Examples are illustrative, but not limiting of the compositions and methods of the present invention. Other suitable modifications and adaptations of a variety of conditions and parameters normally encountered which are obvious to those skilled in the art are within the spirit and scope of this invention.

### EXAMPLES

### Example 1

### Preparation of Cisplatin Microgranules and 3 mm Beads

Calcium sulfate hemihydrate (1 g) is mixed with 120 mg of finely ground cisplatin (cis-diaminedichloroplatinum). To this mixture 0.6 ml of dextran sulfate solution (10%) is added and the slurry is allowed to solidify in bulk-24hr. To produce microgranules the solid matrix-cisplatin is milled and sized to 45-150 microns. Alternatively, the above-mentioned slurry is transferred to a 3 ml syringe and injected into a Teflon mold with spherical holes that are 3 mm in diameter. After 48 hours at room temperature, the mold is split and the beads are removed with a dental explorer under sterile conditions. Both preparations are sterilizable with gamma irradiation (15 kgy).

### Example 2

### Treatment of a Tumor with Cisplatin Beads

Beads (3 mm) are surgically implanted in tumors no more than 3 cm apart to provide optimal coverage. The microgranules are suspended in aqueous 2% HPMC (hydroxypropyl methyl cellulose, USP-2910) and injected with a syringe fitted with a 21 gauge needle. The suspension is prepared by mixing the matrix microgranules (1 g) with the HPMC solution (1 ml) just prior to use. The dosage volume is approximated as follows:
Tumor volume/5=4/15 π r³ for spherical tumors

In other words, the dosage equates to 20% of tumor volume based on a 1/1 suspension of matrix-cisplatin microgranules.

### Example 3

### Preparation of Doxorubicin Microgranules

Doxorubicin hydrochloride (10 mg) is finely ground and mixed with 250 mg of calcium sulfate hemihydrate/calcium stearate (95/5, wt/wt). To this mixture 0.15 ml of 10% dextran sulfate (MW 8,000) solution is added with mixing. The resulting slurry is poured into a tray and allowed to solidify. After 24 hr at room temperature the formulation is milled and sized to yield microgranules (45-150 microns). The formulation is protected from light at all stages.

### Example 4

### Preparation of Paclitaxel Beads

Calcium sulfate hemihydrate/calcium stearate (95/5, wt/wt, 100 mg) is mixed with 3 mg of finely ground paclitaxel. To this mixture 60 μl of PS80 (PEG) solution (10%) is added and the slurry is transferred into a tray and allowed to solidify. After 24 hr at room temperature the formulation is milled and sized to yield matrix microgranules (45-150 microns).

### Example 5

### Preparation of GM-CSF Beads

GM-CSF (0.3 ml at 0.824 mg/ml) is mixed with 30 mg of PVP-10. To this mixture 550 mg of calcium sulfate is added with mixing. The resulting slurry is transferred into a tray and allowed to solidify for an hour at room temperature. The resulting solid is then placed in the refrigerator. After 24 hr the formulation is milled and sized to yield microgranules (45-150 microns).

### Example 6

### Post-Surgical Treatment with Matrix Paclitaxel

Using sonographic guidance, a 21 ga needle is used to introduce matrix-paclitaxel microgranules (suspended in 1% hyaluronic acid) into the breast cavity left by surgery. The remaining cancer cells on the margins of these cavities are eradicated by the treatment.

### Example 7

### Formulation of Oncolysate with an Immunostimulant

Tumor cells are isolated from resected tumor tissue. Treatment in vitro with cisplatin is effected in order to kill 100% of the cells in a 3 hr time period. The cell suspension is mixed with immunostimulant such as GM-CSF containing a matrix polymer such as hyaluronic acid. To each 600 microliters of oncolysate is added 1 g of calcium sulfate hemihydrate. After solidification at room temperature the preparation is milled and sized to 45-150 microns. The resulting microgranules are suspended in a viscous polymer solution for reintroduction to the patient from whom the resected tumor was taken.

It will be readily apparent to those skilled in the art that numerous modifications and additions may be made to the present invention, the disclosed device, and the related system without departing from the invention disclosed.

1. A composition for the controlled release of an antineoplastic agent and an immunostimulant comprising an antineoplastic agent and an immunostimulant dispersed throughout a matrix wherein said composition is the hydration reaction product of an aqueous mixture comprised of:
an inorganic compound capable of undergoing hydration and/or crystallization,
an antineoplastic agent,
an immunostimulant, and
at least one of: a matrix polymer, a complexing agent, and a conditioning agent.

2. A composition as in claim 1 wherein said inorganic compound capable of undergoing hydration and/or crystallization is calcium sulfate hemihydrate.

3. A composition as in claim 1 wherein said matrix polymer is selected from the group onsisting of chondroitin sulfate, hyaluronic acid, dextran sulfate, pentosan polysulfate, polyethylene glycol, polyvinylpyrrolidone, gelatin and fibrinogen.

4. A composition as in claim 1 wherein said matrix polymer is hyaluronic acid.

5. A composition as in claim 1 wherein said antineoplastic agent is selected from the group consisting of carmustin, paclitaxel, doxorubicin, cisplatin, ifosfamide, cytoxan, carboplatin, methotrexate, leuprolide, bleomycin, and 5-flourouracil (5-FU).

6. A composition as in claim 1 wherein said antineoplastic agent is cisplatin.

7. A composition as in claim 1 wherein said antineoplastic agent is 5-FU.

8. A composition as in claim 1 wherein said immunostimulant is GM-CSF.

9. A composition as in claim 1 wherein said antineoplastic agent is an apoptosis inducer.

10. A composition as in claim 1 wherein said delivery system is in the form of matrix beads or microgranules, or a slurry.

11. A composition as in claim 1 wherein said antineoplastic agent and said immunostimulant are each formulated in a separate matrix and the matrices are mixed together.

12. A composition for the controlled release of cisplatin and GM-CSF comprising cisplatin and GM-CSF dispersed throughout a calcium sulfate dihydrate matrix wherein said composition is the hydration reaction product of an aqueous mixture comprised of:
calcium sulfate hemihydrate,
cisplatin,
GM-CSF, and
a matrix polymer.

13. A composition for the controlled release of a radiation potentiator comprising a radiation potentiator dispersed throughout a matrix wherein said composition is the hydration reaction product of an aqueous mixture comprised of:
an inorganic compound capable of undergoing hydration and/or crystallization,
a radiation potentiator, and
at least one of: a matrix polymer, a complexing agent, and a conditioning agent.

14. A composition as in claim 13 further comprising a radioisotope.

15. A composition as in claim 13 wherein said radiation potentiator is paclitaxel.

16. A composition as in claim 14 wherein said radioisotope and said radiation potentiator are each formulated in a separate matrix.

17. A composition for the controlled release of paclitaxel comprising paclitaxel dispersed throughout a calcium sulfate dihydrate matrix wherein said composition is the hydration reaction product of an aqueous mixture comprised of:
calcium sulfate hemihydrate,
paclitaxel, and
a matrix polymer.

18. A composition as in claim 13 wherein said delivery system is in the form of matrix beads or microgranules, or a slurry.

19. A method of treating a solid tumor in a mammal comprising:
administering to said mammal a resorbable delivery system for sustained release of i) an antineoplastic agent and ii) an immunostimulant

20. A method as in claim 19 wherein said administering is done by injecting said delivery system intra-tumorally or peri-tumorally.

21. A method as in claim 19 wherein said administering is done by injecting said delivery system into the tumor vasculature.

22. A method as in claim 19 wherein said administering is done by injecting said delivery system into a cavity left by tumor resection.

23. A method as in claim 19 wherein said delivery system is in the form of matrix beads or microgranules.

24. A method as in claim 19 wherein said administering step comprises administering said antineoplastic agent and said immunostimulant in separate sustained release delivery systems.

25. A method as in claim 19 wherein said antineoplastic agent is selected from the group consisting of carmustin, cisplatin, paclitaxel, and doxorubicin.

26. A method as in claim 19 wherein said immunostimulant is selected from the group consisting of LPS, BCG, IL-1, IL-2, GM-CSF, and TNF-alpha.

27. A method as in claim 19 wherein said immunostimulant is IL-2.

28. A method as in claim 19 wherein said immunostimulant is GM-CSF.

29. A method as in claim 19 wherein said antineoplastic agent is cisplatin and said immunostimulant is GM-CSF.

30. A method as in claim 19 wherein said antineoplastic agent is an apoptosis inducer.

31. A method as in claim 19 wherein said immunostimulant is a genetic construct that encodes an immunostimulant.

32. A method as in claim 19 wherein said antineoplastic agent is cisplatin.

33. A method as in claim 19 wherein said tumor is a tumor associated with a cancer selected from the group consisting of: skin cancer, breast cancer, head and neck cancer, gynecological cancer, urological and male genital cancer, bladder cancer, prostate cancer, bone cancer, cancers of the endocrine glands, cancers of the alimentary canal, cancers of the major digestive glands/organs, CNS cancer, and lung cancer.

34. A method as in claim 19 wherein said tumor is selected from the group consisting of prostate, breast, brain, bladder, head and neck tumors.

35. A method as in claim 19 wherein said delivery system is administered by injection.

36. A method as in claim 19 wherein said delivery system is administered by cannula or endoscope.

37. A method as in claim 19 wherein said administering also includes administering systemically the same or a different antineoplastic agent that is administered locally.

38. A method as in claim 19 wherein said administering includes administering the delivery system locally and administering systemically an antineoplastic agent and/or the immunostimulant.

39. A method as in claim 19 wherein said administering includes administering a delivery system including an apoptosis inducer and an immunostimulant.

40. A method as in claim 19 wherein said resorbable delivery system for sustained release of an antineoplastic agent and an immunostimulant comprises a composition for the controlled release of an antineoplastic agent and an immunostimulant comprising an antineoplastic agent and an immunostimulant dispersed throughout a matrix wherein said composition is the hydration reaction product of an aqueous mixture comprised of:
an inorganic compound capable of undergoing hydration and/or crystallization,
an antineoplastic agent,
an immunostimulant, and
at least one of: a matrix polymer, a complexing agent, and a conditioning agent.

41. A method as in claim 40 wherein said inorganic compound is calcium sulfate hemihydrate.

42. A method of treating a solid tumor in a mammal comprising:
a) administering to said mammal a resorbable delivery system for sustained release of a radiation potentiator, and
b) irradiating said tumor.

43. A method as in claim 42 wherein said administering is done intra-tumorally or peri-tumorally.

44. A method as in claim 42 wherein said administering is done by injecting said delivery system into the tumor vasculature.

45. A method as in claim 42 wherein said administering is done by injecting said delivery system into a cavity left by tumor resection.

46. A method as in claim 42 wherein said delivery system is in the form of matrix beads or microgranules.

47. A method as in claim 42 wherein said radiation potentiator is selected from the group consisting of paclitaxel, nimorazole, metronidazole, and 5,6-dimethylxanthenone-4-acetic acid.

48. A method as in claim 42 wherein said radiation potentiator is paclitaxel.

49. A method as in claim 42 wherein said radiation potentiator is metronidazole.

50. A method as in claim 42 wherein said irradiating step comprises administering a resorbable delivery system for sustained release of a radioisotope.

51. A method as in claim 50 wherein said radioisotope is selected from the group consisting of Pd-103, I-125 and Ir-192.

52. A method as in claim 42 wherein said tumor is a tumor associated with a cancer selected from the group consisting of: skin cancer, breast cancer, head and neck cancer, gynecological cancer, urological and male genital cancer, bladder cancer, prostate cancer, bone cancer, cancers of the endocrine glands, cancers of the alimentary canal, cancers of the major digestive glands/organs, CNS cancer, and lung cancer.

53. A method as in claim wherein said tumor is selected from the group consisting of prostate, breast, bladder, brain, head and neck tumors.

54. A method as in claim 42 wherein said delivery system is administered by direct injection.

55. A method as in claim 42 wherein said delivery system is administered by cannula or endoscope.

56. A method as in claim 42 wherein said administering includes administering the delivery system locally and administering systemically the same or a different radiation potentiator.

57. A method as in claim 42 wherein said resorbable delivery system for sustained release of an antineoplastic agent and an immunostimulant comprises a composition for the controlled release of a radiation potentiator comprising a radiation potentiator dispersed throughout a matrix wherein said composition is the hydration reaction product of an aqueous mixture comprised of:
an inorganic compound capable of undergoing hydration and/or crystallization,
a radiation potentiator, and
at least one of: a matrix polymer, a complexing agent, and a conditioning agent.

58. A method as in claim 57 wherein said inorganic compound is calcium sulfate hemihydrate.

59. A method of treating a tumor in a mammal comprising:
a) treating ex vivo tumor cells from said mammal with an antineoplastic agent, and
b) administering to said mammal said tumor cells treated in step a) and
c) administering to said mammal a resorbable delivery system for sustained release of an immunostimulant.

60. A method as in claim 59 wherein said resorbable delivery system for sustained release of an immunostimulant includes tumor cells treated in step a), and an immunostimulant.

61. A method as in claim 59 wherein said immunostimulant is GM-CSF

62. A method as is claim 59 wherein said delivery system is in the form of microgranules.

63. A method as in claim 59 wherein said administration is by injection.

64. A method as in claim 59 wherein said administering is done by injecting said delivery system intra-tumorally or peri-tumorally.

65. A method as in claim 59 wherein said administering is done by injecting said delivery system into the tumor vasculature.

66. A method as in claim 59 wherein said administering is done by injecting said delivery system into a cavity left by tumor resection.

67. A method as in claim 59 wherein said administration is by subcutaneous injection.

68. A method as in claim 59 wherein said immunostimulant is of microbiological origin.
